(19) United States
(12) Patent Application Publication
(10) Pub. No.: US 2011/0028937 A1
(43) Pub. Date: Feb. 3, 2011

(54) AUTOMATED FLUID FLOW CONTROL SYSTEM

(75) Inventors: Benjamin G. Powers, Portsmouth, NH (US); Jeffrey A. Carlisle, Stratham, NH (US); Lawrence M. Kuba, Nashua, NH (US)

Correspondence Address: SCOTT C. RAND, ESQ.;MCLANE, GRAF, RAULERSON & MIDDLETON, PA, 900 ELM STREET, P.O. BOX 326, MANCHESTER, NH, 03105-0326, US

(73) Assignee: FLUIDNET CORPORATION, Amesbury, MA (US)

(21) Appl. No.: 12/906,077

(22) Filed: Oct. 16, 2010

Related U.S. Application Data
(63) Continuation-in-part of application No. 12/280,894, filed on Aug. 27, 2008.
Application No. 12/280,894 is a continuation-in-part of application No. 12/280,869, filed on Aug. 27, 2008.
(60) Provisional application No. 60/777,193, filed on Feb. 27, 2006.
(60) Provisional application No. 60/777,193, filed on Feb. 27, 2006.

(51) Int. Cl. A61M 5/168

(52) U.S. Cl. 604/500; 604/67

(57) ABSTRACT

A system for controlled delivery of medicinal fluid includes a fluid pathway assembly defining a fluid pathway and including means for calculating a first calculated fluid flow rate using gas laws. The fluid pathway assembly has an inline flow sensor element received within the fluid pathway movable in response to fluid flowing in the fluid pathway. A flow control device is removably attached to the fluid pathway assembly and has a sensor for sensing a position of the inline flow sensor element in the fluid pathway, the position of the inline flow sensor element being representative of a second calculated fluid flow rate. The fluid pathway assembly includes a variable flow resistor adjustable to regulate a rate of fluid flow in the fluid pathway assembly. A drive mechanism attached to the flow control device is operably coupled to the variable flow resistor when the flow control device is attached to the fluid pathway assembly. The variable flow resistor is adjustable by the drive mechanism to achieve a target flow rate when the first calculated flow rate and/or the second calculated flow rate differs from the target flow rate.

### RELATED APPLICATIONS

This application claims priority, as a continuation-in-part type application, under 35 U.S.C. §120 to U.S. patent application Ser. No. 12/280,894, filed Aug. 27, 2008, now pending, which is a 371 of application No. PCT/US07/04945, filed Feb. 27, 2007, which claims priority to U.S. provisional patent application Ser. No. 60/777,193, filed on Feb. 27, 2006.

This application also claims priority, as a continuation-in-part type application, under 35 U.S.C. §120 to U.S. patent application Ser. No. 12/280,869, filed Aug. 27, 2008, now pending, which is a 371 of application No. PCT/US07/02039, filed Jan. 23, 2007, which claims priority to U.S. provisional patent application Ser. No. 60/777,193, filed on Feb. 27, 2006.

Each of the aforementioned applications is incorporated herein by reference in its entirety.

### BACKGROUND

The present disclosure relates to intravenous infusion therapy. More specifically, the disclosure relates to a system, components of the system, and methods associated with the system for organizing the fluid flow for applications which require an accommodation of a broad flow rate range, a wide range of input and output pressures, and a wide range of delivered fluid viscosities, such as those seen with Intravenous (IV) infusion therapy.

Conventionally, healthcare providers have had three technical options for intravenous infusions. Many intravenous infusions are controlled by manually adjusting a resistance in the flow path between a fluid source and the patient, based on the operator's observation of the rate of drips formed within a chamber in line with the fluid flow. The flow rate range that can be controlled with this method is limited by the relatively large and fixed size of the drops and the relatively low reliability of the human operator to accurately compute the flow rate. This method is critically flawed by virtue of the fact that it requires a human observer to maintain an accurate and consistent flow rate. In many circumstances, a trained human observer is not available. This manual method also lacks an important ability to electronically record and communicate the results of the infusion.

A relatively small number of infusions are controlled with the use of a fixed volume of liquid under a fixed amount of pressure and a fixed resistance, providing a fixed flow rate. Unfortunately, the fixed rate and fixed fluid volume do not provide the flexibility required for most infusions. Similar to a manual infusion, this method does not provide the opportunity to electronically record the results of the infusion.

Because of the strong requirement for more precise control of flow rate, flexibility of fluid volumes, and the desire to keep track of the flow information, many infusions are controlled using a positive displacement fluid pump. These large volume positive displacement devices are generally of the peristaltic or reciprocating piston type. Both types come at a price of complexity, size, weight, limited battery life, and significant financial cost. Early versions of positive displacement pumps created a new hazard for patients in what was known as “runaway infusion,” where the highly controlled fluid flow was suddenly uncontrolled when a door or other containment mechanism on the pump was released. In response to this undesirable feature, pumps were later required to incorporate “flow stop” mechanisms, so that the flow rate would stop entirely if the fluid tubing were removed from the flow control device. Unfortunately, the cessation of flow is sometimes as hazardous to patients as a sudden increase. Another unintended consequence of positive pumping systems is the possibility of infusing lethal amounts of air into a patient. This possibility did not exist with low pressure gravity infusions. As a result, positive displacement pumps have incorporated air detection systems to prevent this hazard, yet these alarm systems are the source of very significant nuisance alarms, resulting in operator inefficiency and patient anxiety.

The present disclosure recognizes the safety advantages inherent in a low pressure infusion, the need to accurately control flow, and the necessity of modern healthcare environments to have infusion data electronically available.

### SUMMARY

The disclosure is directed to an medicinal fluid administration apparatus and method for using this apparatus, comprising a fluid pathway assembly and a flow control device wherein fluid flowing through the fluid flow system is controlled via closed loop quasi-static adjustment of in-line pressure based resistance in combination with a low pressure pneumatic pump element. This sensor-based infusion platform (SIP) utilizes wireless communication to a network to maintain device software and dataset integrity, broadcast alarms, and record infusion status information.

These and other features of the disclosure, including various novel details of construction and combinations of parts, will now be more particularly described with reference to the accompanying drawings and pointed out in the claims. It will be understood that the particular device embodying the invention is shown by way of illustration only and not as a limitation of the invention. The principles and features of this disclosure may be employed in various and numerous embodiments without departing from the scope of the invention.

### BRIEF DESCRIPTION OF THE DRAWINGS

These and other features, aspects, and advantages of the apparatus and methods of the present invention will become better understood with regard to the following description, appended claims, and accompanying drawings where:

FIG. 1a is a rear view of the preferred embodiment of the Flow Control Device (controller) with the Fluid Path (disposable) installed as would be to deliver an infusion;

FIG. 1b shows the two major assemblies of the embodiment herein—the Flow Control Device or controller with a Fluid Path (disposable administration set) installed in the pocket in the rear of the of the device;

FIG. 2 is a rear perspective view of the flow control device showing the interface to the cassette;

FIG. 3 shows an exploded view of the controller;

FIG. 4 shows an assembled disposable including a cassette and tubing;

FIG. 5a shows a section view of the intermediate pumping chamber;

FIG. 5b shows the check valves and fluid path to the intermediate pumping chambers;

FIG. 6 shows a cross sectional view of the variable resistance device;

FIG. 7 shows a preferred embodiment of the flow sensing element;

FIG. 8a shows a graph of the sensor output peaks formed when the element focuses and transmits light to the detector;

FIG. 8b shows a graph of a sensor output peak with the flow object;

FIG. 8c shows a graph of a sensor output peak with one LED illuminated; and

FIG. 9 shows the IV pole bracket mount for the controllers.

### DETAILED DESCRIPTION OF THE PREFERRED EMBODIMENTS

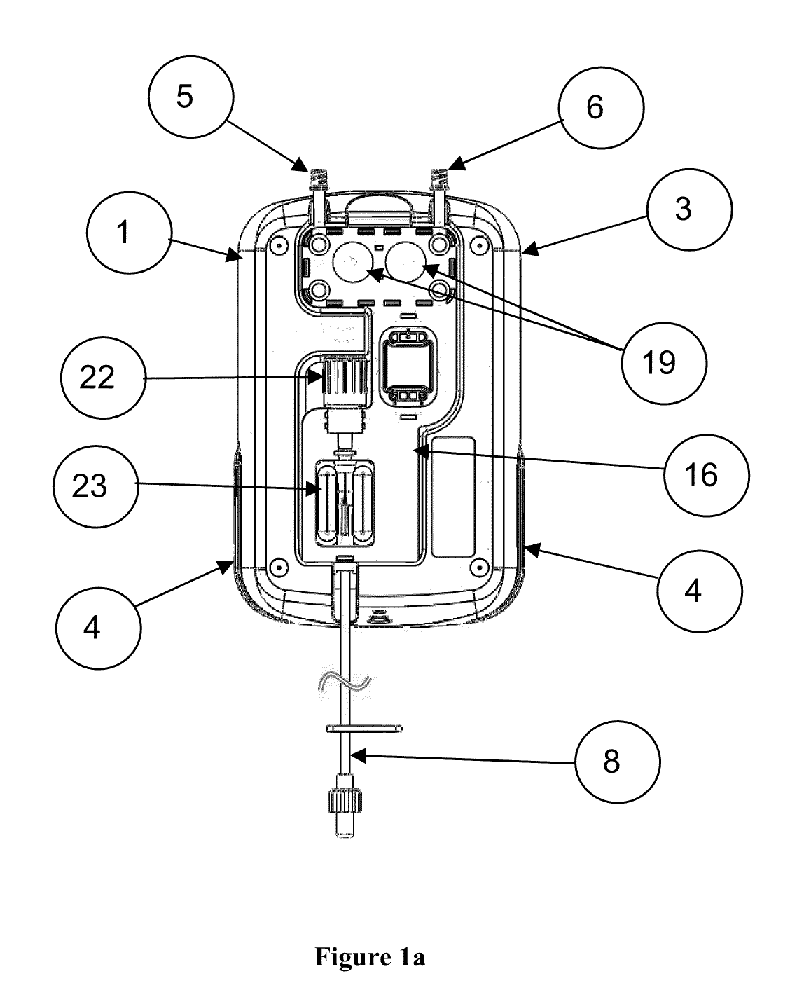

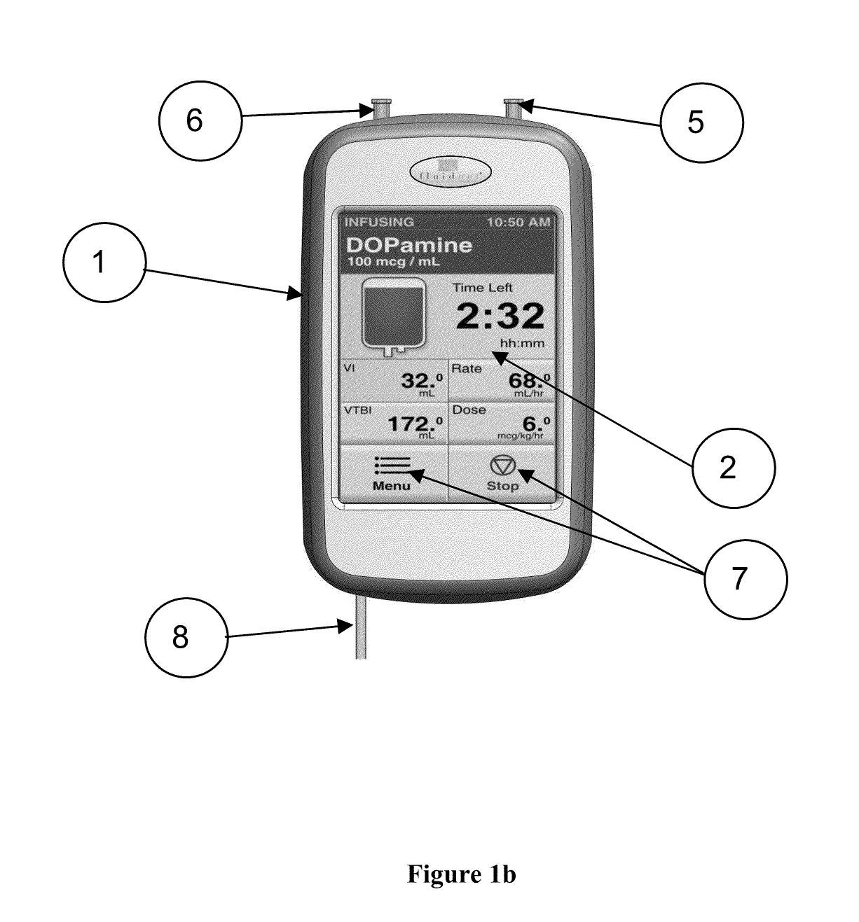

Referring to the drawings, wherein like reference numerals are used to indicate like or analogous components throughout the several views, FIGS. 1a and 1b depicts an exemplary volume and flow measurement system in accordance with an exemplary embodiment of the present invention. The full sensor based infusion platform system includes a disposable, a controller, an IV pole mounting bar, and a networked computer.

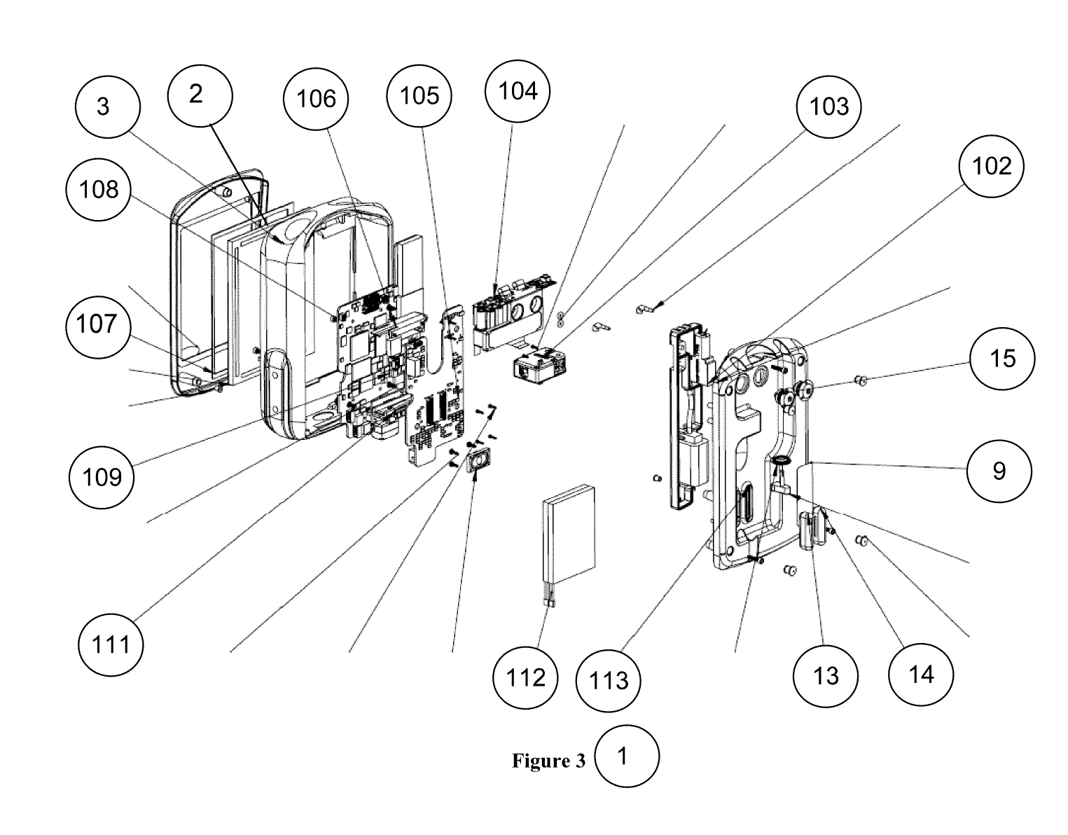

Referring now to FIGS. 1a and 1b, where an exemplary embodiment of the present invention is shown, FIG. 1a is a rear view of the controller with a disposable installed and FIG. 1b shows a front view of the controller with a disposable installed. The controller 1 includes a display 2, which is preferably an LCD display and more preferably a color LCD display with a touch-sensitive input device, such as a capacitive or resistive touch screen overlay 107 (see FIG. 3). Alternative user input devices are also contemplated, such as a keypad or keyboard, mouse, trackball, touchpad, joystick, or combinations thereof as would be understood by persons skilled in the art.

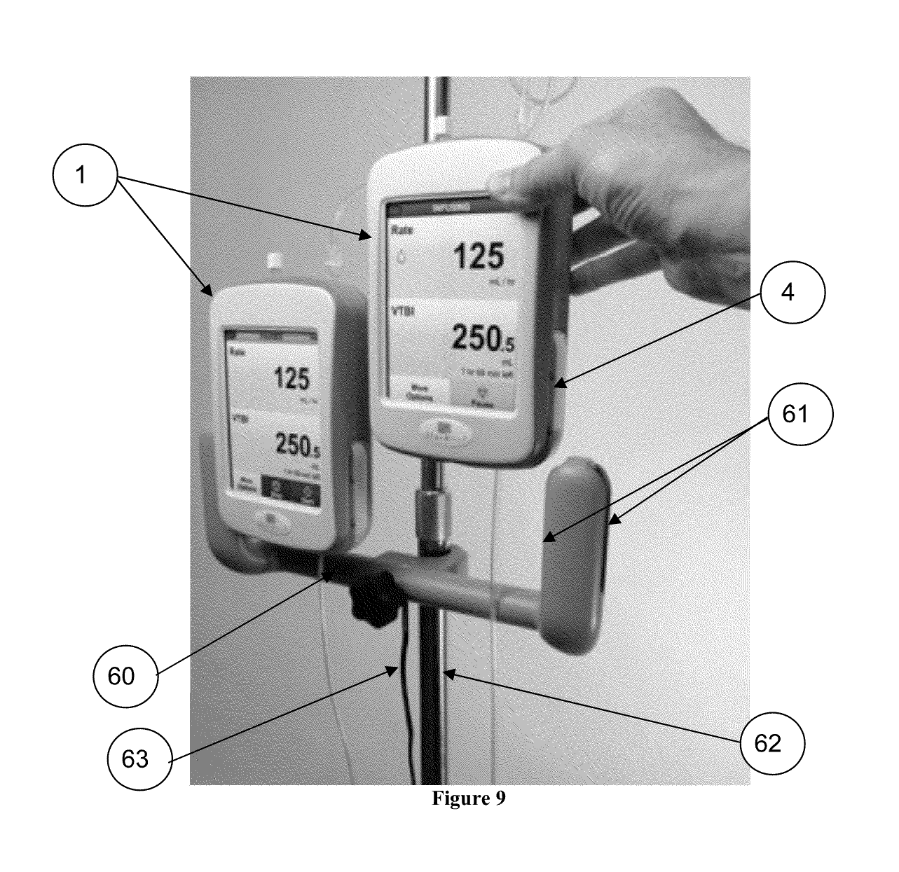

The display 2 is housed in a case or housing 3, e.g., formed of rigid plastic. The controller includes an interface 4 to the pole mount device 60 (see FIG. 9), which both mechanically secures the controller 1 to the IV pole 62 (see FIG. 9). The pole mount 60 may also include a charger for charging the internal batteries or battery pack in the controller, e.g., via charging contacts which are aligned with and electrically couple charging contacts on the controller, or alternatively via induction, when the controller is placed in the mount. Preferably, the charger can charge the internal batteries on either side of the device. The case 3 may include ergonomically designed finger grips or recesses around the circumference to facilitate gripping of the device and may further include a pliable insert either removably or permanently attached to the outer housing 3, for example, via over-molding, co-molding, or otherwise attaching a flexible or resilient material over the rigid shell 3 to further enhance the grip ability of the device.

The inlets 5 and 6 and outlet 8 tube of the disposable are also visible in FIG. 1b. The primary inlet 5 connects the primary fluid source (not shown) containing a volume of fluid to be delivered to the device through a standard luer fitting as is known in the art. Fluid travels through the cassette housed in the rear of the device and then flows to the patient connection through the outlet 8.

The secondary inlet 6 allows a second fluid to be connected to a device independently of and without affecting the current infusion, and then the user can program the device with the second fluid delivery parameters, including start time. At the secondary infusion programmed start time, the controller 1 will temporarily pause delivery of the primary infusion, deliver the secondary infusion per the programmed parameters, and then resume the primary infusion. Other infusion devices on the market require the user to physically hang the second fluid source higher than the first fluid source such that the static pressure of the higher source determines which fluid is delivered. When the hydrostatic head height of second fluid source is not sufficiently higher than that of the primary source, the pump will deliver a mix of both primary and secondary fluids depending on the relative static pressures of the sources, thus not delivering the secondary fluid at the rate—and therefore not delivering the secondary fluid at the desired effective dose—prescribed. This issue, i.e., dependence on the user to manipulate both primary and secondary bag heights, is overcome with this disclosure, as the preferred embodiment will deliver the secondary infusion as programmed independent of the static pressure of the fluid sources.

Features of the disposable administration set (“disposable”) 16, and specifically, the cassette portion of the disposable can be seen in FIG. 1a, including the variable flow resistor 22, the flow sensor 23, the flow sensor 23, and the intermediate pumping chambers 19. The variable flow resistor 22 can be automatically adjusted by the controller to match the sensed flow rate with the program flow rate. The flow sensor 23 includes a flow element in the fluid path that moves in response to flow rate and provides the system with both a signal representative of flow rate, but also has a unique signal when air is passing through the sensor. The intermediate pumping chambers 19 pneumatically couple to the controller and act as both pneumatic pumps and additional flow sensors.

FIG. 1b shows the touch-screen display 2 which displays a graphical user interface that is divided into several sections. These sections include information and status displays, status displays that include virtual navigation buttons, and navigation buttons 7. Color and shading of the user interface intuitively show the user where more information is available. The user can touch an onscreen object such as an icon or button to navigate to pages (e.g., which may be arranged in a hierarchical fashion) with more information and change or update the program parameters if needed.

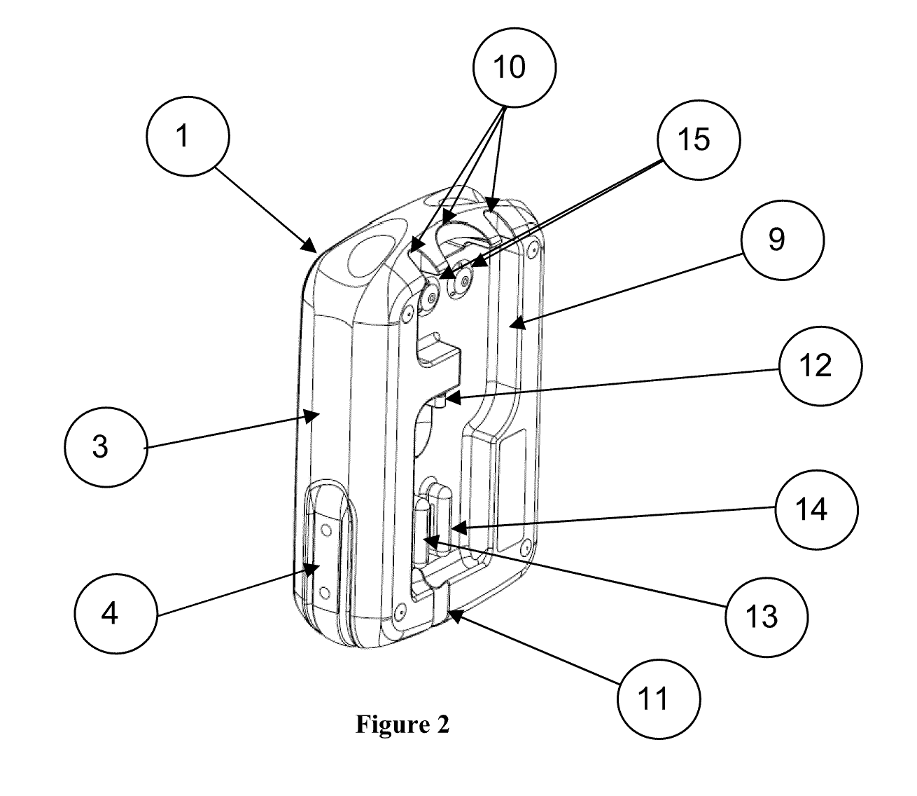

Referring now to FIG. 2, the controller 1 is shown generally from the back and side, where the interface to the disposable is visible. The rear housing 9 is configured to guide the user in proper placement of the disposable into the controller. The asymmetric recess in the rear housing 9 together with recesses 10, 11 provided to allow passage of the primary and secondary inlets 5, 6 and the outlet tube 8, respectively, are three of several features that key the disposable to the controller, thereby preventing the disposable 16 from being installed incorrectly. A rib or spline 12 interlocks with and manipulates the variable flow resistor and is positioned to only allow insertion of a disposable only when the resistor is in the fully closed position (thus preventing uncontrolled flow). Once engaged, the spline 12 does not allow the disposable to be removed from the controller without again fully closing the variable flow resistor.

The light source array 13 and the optical detector 14 are positioned to allow the movable flow element in the disposable to be located between them. When in use, the light source array 13 can preferentially illuminate specific segments of the array, e.g., based on the anticipated location of the flow element, thus enhancing the ability of the optical detector 14 to accurately sense the location of the flow element and saving power to maximize battery run time. The pneumatic interface 15 to the intermediate pumping chambers (IPC's) of the disposables include o-ring seals which help both guide the nipple on the disposable and seal the connection.

Referring now to FIG. 3, where more details of the controller 1 architecture can be seen, the pneumatic interface 15 connects to the manifold 104, housing the valves and sensors, and connecting the pump chamber assembly 102. Pressure sensors in the manifold 104 allow the system to accurately measure pressure in each of the intermediate pumping chambers in the disposable as well as in a calibration chamber of known volume. Isolating the calibration chamber of known volume from the intermediate pumping chambers using the valves in the manifold 104, measuring the pressure present in each chamber, then combining the calibration chamber to an intermediate pumping chamber by opening a valve and measuring the resulting pressure allows the system to calculate the volume of fluid in the intermediate pumping chamber using ideal gas laws. As used herein, the term “ideal gas law” is intended to encompass not only the equation PV=nRT, but also special cases of this law, such as Boyle's Law and Charles' Law. The fluid flow rate is calculated by periodically calculating the volume of fluid entering and leaving the intermediate pumping chambers over time.

The pump chamber assembly 102 includes the pumps and chambers creating a positive pressure source and a negative pressure source. These pressure sources are connected through the manifold 104 to the intermediate pumping chambers of the disposable. As negative pressure is connected to an intermediate pumping chamber, fluid is drawn from the fluid source. As positive pressure is connected to an intermediate pumping chamber, fluid is expelled from the chamber. Controlling the pressures in each of the sources allows the system to compensate for changes in source height and in changes in outlet back pressure. Controlling the timing of the pressure changes allows the system to change the fluid flow rate through the system.

A second means of control of fluid flow through the system is accomplished by the inclusion of a variable flow fluid resistor within the fluid flow path that can be manipulated by the variable resistor drive mechanism 103. The drive mechanism 103 includes a motor and gear mechanism that output torque to a spline 12 (see FIG. 2) that couples with the variable flow resistor on the disposable. As the spline rotates over its 300-degree range of motion, it moves the variable resistor from fully closed to fully open. The resistor is designed to provide a logarithmic response throughout its range of motion, yielding an effective control over a four order of magnitude range (e.g., 0.1-1000 ml/hour) of the system.

The control board assembly 105 including a processor, microprocessor, or the like, and associated electronics executes the fluid delivery programs sent to it by the user interface (UI) board assembly 106. The control board assembly 105 also manages inputs from temperature sensors, an external pressure sensor, the intermediate pump chamber pressure sensors, and the flow sensor; determines and executes changes in pneumatic pressure and resistance settings to match the measured flow rate to the programmed flow rate and sends infusion status updates to the UI board assembly 106. The UI board assembly 106 includes a three axis accelerometer for motion sensing as well as sensors for monitoring the ambient noise level. This data, including the temperature and pressure signals collected and managed on the control board assembly 105, allows the pump to be situationally aware.

The UI board assembly 106 drives the display 2 and manages the user interface, allowing users to program new infusions, change the parameters of existing infusions, and view the history and status of infusions run on the device. The UI board assembly 106 also manages communication with the control board assembly 105 and communications to networked computers. The UI board assembly 106 may include one or more wireless, e.g., radio frequency (RF) or infrared (IR) transceivers, and in the preferred embodiment includes both 802.11 (WIFI) and 802.15 (ZIGBEE) radios 108 and 109, respectively, to enable wireless network communications. Network communication enables the device to send infusion status information to populate electronic medical records, e.g., stored in a network database or remotely located database) and alarm notifications to page the caregiver. Network communications also allows the device to receive updated infusion datasets and software updates.

If the ZIGBEE 109 network is installed in the hospital or other use environment, the device becomes location aware, and the location of the device can be included in all messages. Since location of the device is often associated with a patient, the device can assist the user in identifying the patient to whom the device is attached. Additionally, ZIGBEE networks—because they are mesh networks—allow the software to warn a caregiver if the same medication in the same location is already being given to the same patient. In acute cases, some patients may be connected to up to 12 infusion devices. Devices currently on the market warn the caregiver if the same drug is already being infused only if it is on the same device as the one being programmed, which can lead to poor outcomes for the patient.

The ZIGBEE networked advantage of the preferred embodiment herein is to improve safety by having communication between all devices within a specific location, coordinating infusions and communication to caregivers. A further benefit of a ZIGBEE network is the ability to use ZIGBEE frequency RFID devices on caregivers. When a caregiver walks near a ZIGBEE device with the RFID device, the system recognizes and records that that caregiver is associated with a device. Associating caregivers, patients, and infusions helps provide complete electronic documentation. When a caregiver chooses to program a new infusion, the caregiver selects the drug to be infused, e.g., by viewing it on display 2 and using the touch screen 107 to choose it from a dataset on the device, or by using the controller's bar code imager 111 mounted on the UI board assembly 106 and imaging a bar code, e.g., located on the source of fluid to be infused, through a window in the bottom of the case 3. The bar code imager 111 preferably is of the type that decodes one and two dimensional bar codes and can be used for patient identification, drug identification, drug infusion programming, and caregiver identification. The depicted controller 1 has a dual battery pack 112, providing system redundancy and extended runtime.

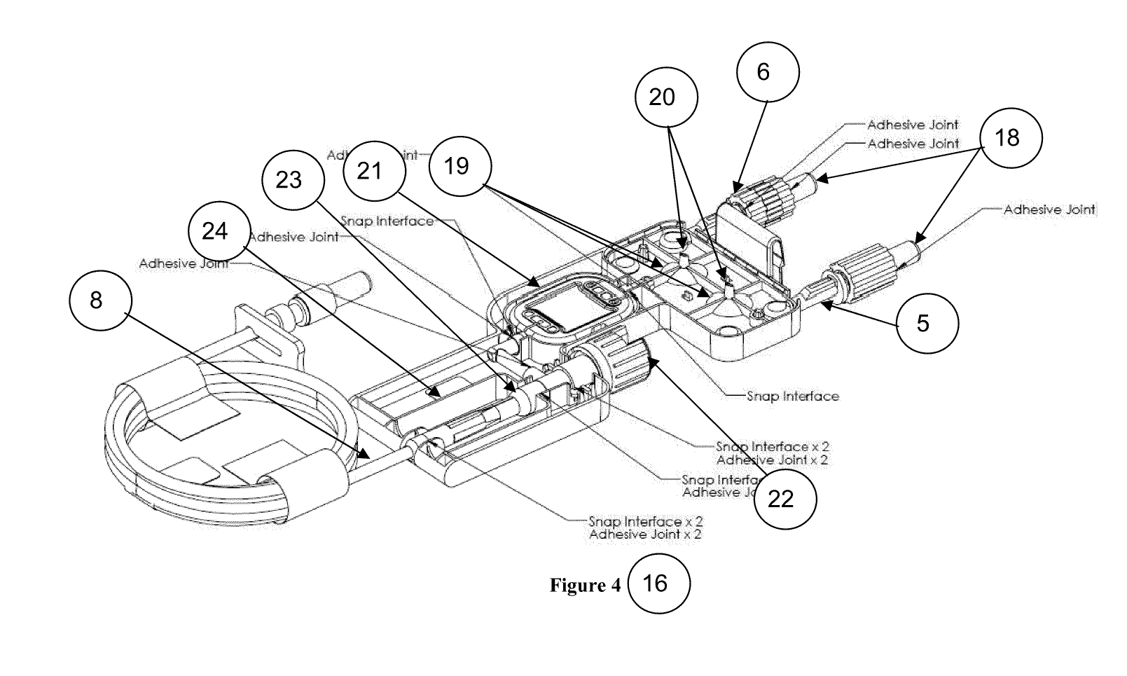

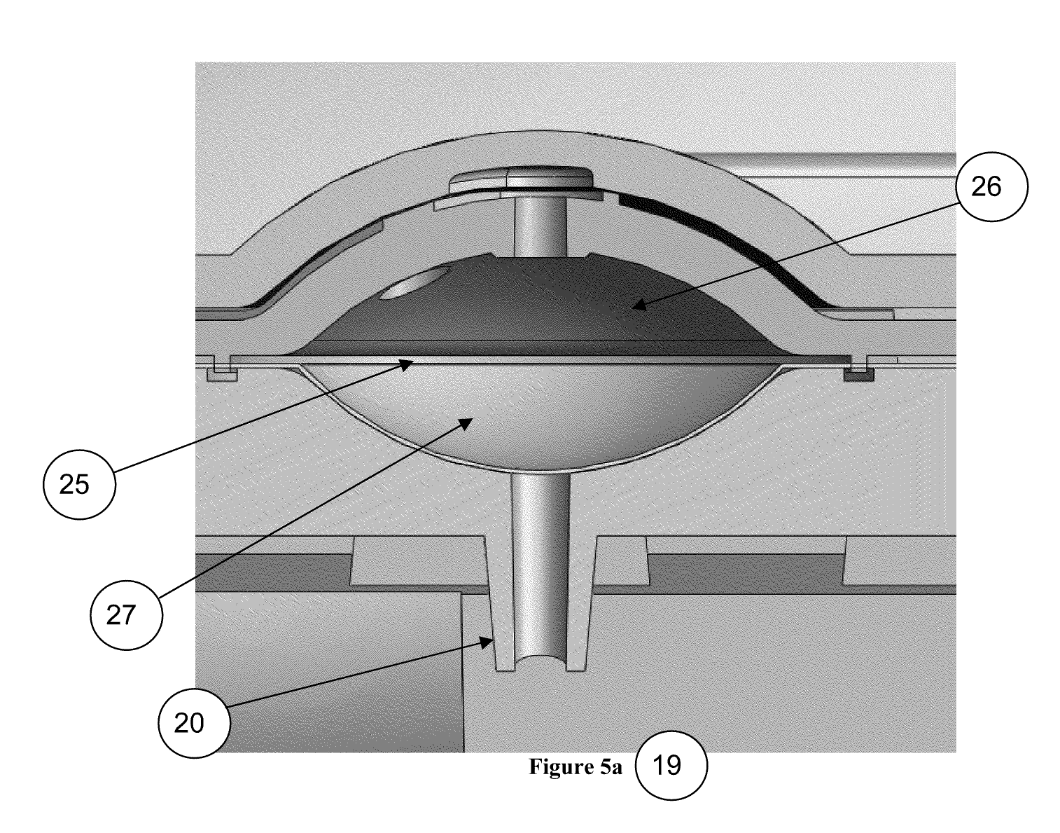

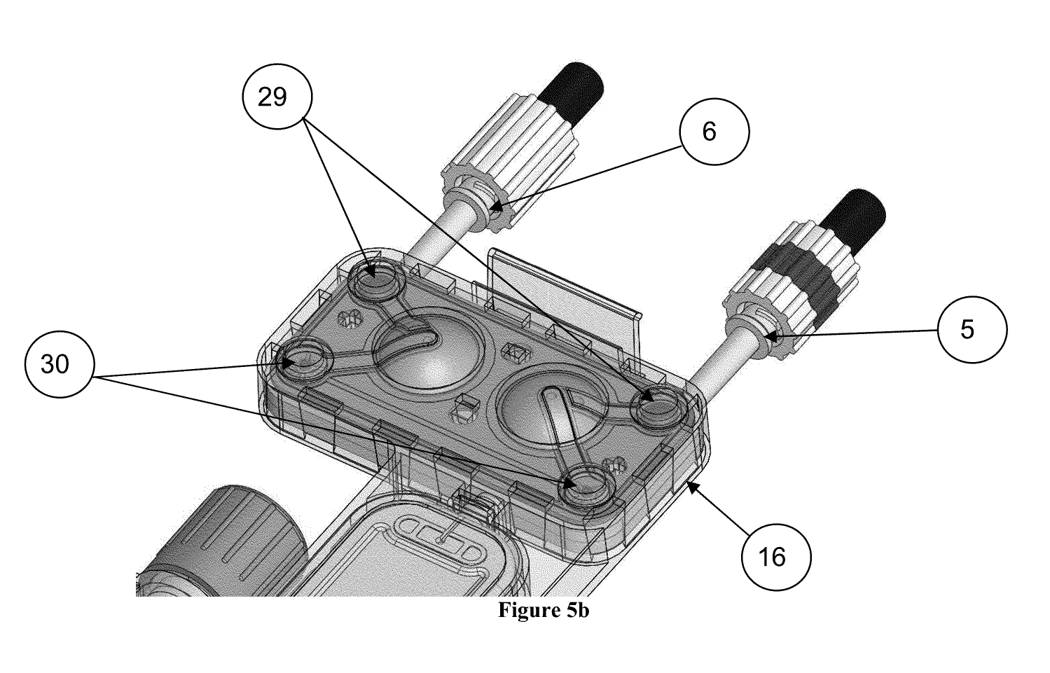

Referring now to FIGS. 4, 5a and 5b, the disposable 16 includes an inlet tube which attaches to the inlet. The disposable 16 may also include a drip chamber and spike (not shown), which can either be used to deliver a gravity infusion, or, in combination with the controller 1, can be used to deliver a sensor based infusion. The disposable 16 has a primary inlet 5 and a secondary inlet 6, both shown with vented caps 18. Fluid from the primary or secondary fluid source flows through the respective inlets 5 or 6 and enters the intermediate pumping chambers 19 through a corresponding one of the one-way or check valves 29. The intermediate pumping chambers 19 are divided by a flexible membrane 25 into two separate volumes 26 and 27.

The fluid entering the chamber flows into volumes 26, and a gas (air) occupies volume 27. The volume 27 that is filled with gas is separated from the fluid in the fluid volume 26 by the flexible membrane 25 and has a port 20 shaped like a nipple, which couples to the pneumatic interface 15 of the controller 1.

When controller 1 applies negative pressure through port 20 to the gas filled volume 27, the flexible membrane moves toward port 20 drawing fluid from the fluid source to fill the chamber. When the controller applies positive pressure through the port 20 to the gas filled volume 27, the flexible membrane is driven from port 20 displacing fluid from the chamber. When all fluid is driven from volume 26, the flexible membrane 25 forms a seal against the fluid outlet of chamber 19. If positive pressure is left in volume 27, the outlet sealed by the membrane 25 will prevent fluid flow when flow is not desired.

Check valves 29 and 30 for each of the primary and secondary flow channels ensure that fluid flows only from the fluid source to the outlet of the disposable 16. The valves 29 prevent fluid in the volume 26 from exiting the volume 26 via the respective inlets 5, 6, e.g., when a positive pressure is applied to the gas volume 27 during operation Likewise, the valves 30 prevent fluid downstream of the intermediate pumping chamber from being drawn back into the pumping chamber, e.g., when a negative pressure is applied to the gas volume 27 during operation.

Pressure sensors in the controller can determine the pressure in the gas filled volume 27 of the intermediate pumping chamber 19. By sensing the pressure in the gas filled volume and the pressure in a known calibration volume in the manifold 104 and then combining the volumes and measuring the resultant pressure of the combined volumes, the volume of gas in the intermediate pumping chamber can be calculated using the ideal gas law.

If the volume of the rigid IPC is precisely known, it is possible to infer the volume of liquid in the IPC. However, in some instances, e.g., due to manufacturing tolerances variations, it is preferable not to presume that the IPC volume is precisely known and to monitor the flow rate of liquid out of the system using a volume calculation which does not require knowledge of the IPC volume and/or liquid volume. In the preferred embodiment, flow rate is determined by measuring an initial volume of compressible gas in the volume 27 and then monitoring pressure decay in the chamber 27 over time. In reducing the system of the present embodiment to practice, a 500 micro liter combined volume 26 and 27 of the intermediate pumping chambers 19 was selected as being advantageous for both high and low flow rates in that it accommodates the need for flow continuity in the low flow range (e.g., -less than 1 ml/hour) as well as the need to be able to deliver rapid infusions (e.g., greater than 1000 ml/hour), although other volumes are contemplated.

It can be seen with this design how the system described herein can pause delivery of the primary fluid entering the primary port 5 and being delivered at a primary flow rate, deliver a secondary fluid from the secondary input port 6 at a second flow rate, and then resume delivery of the primary fluid without the need to depend on the user changing the bag heights or otherwise needing to remember to connect, move or otherwise manipulate the primary infusion setup. This arrangement prevents secondary fluid flowing into the primary infusion source, or drawing from both secondary and primary fluid sources at an unknown mix rate, both common occurrences with other systems if the caregiver is not meticulous in system configuration.

Fluids leaving the intermediate pumping chambers 19 flow through an air-elimination filter 21. Many systems in use combine a peristaltic mechanism with a silicone pumping member. Silicone is semi permeable to air and when combined with the high pressures typical of a peristaltic device, air becomes entrained in the fluid being infused. Ultrasonic sensors positioned downstream of the pumping mechanism are employed in those devices to transmit through the tubing of the disposable looking for evidence of air. Those devices have been the source of nuisance (false) alarms and the ensuing wasted time, disposables, and medicinal fluids as caregivers have attempted to remedy constant alarms by changing sets.

This disclosure overcomes those issues by eliminating a high pressure pumping member, which is the root causes of those alarms, instead using low pressure, impervious membranes and incorporation of an air elimination filter. As will be seen, the fluid flow sensor output has a characteristic signature for air and can therefore give an additional layer of safety without an inherent false positive (nuisance) alarm. Fluid passing through the air elimination filter 21 enters the inlet 30 of the variable flow resistor 22.

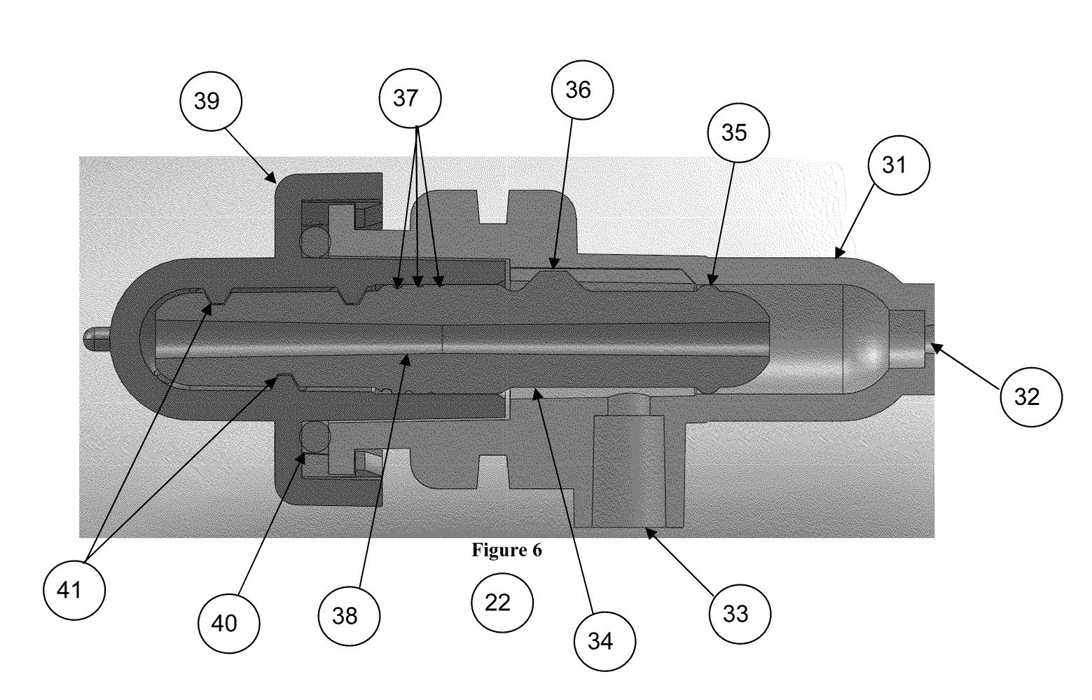

Referring now to FIG. 6, when the disposable is used for a gravity infusion (i.e., without the use of the controller), the cap 39 can be manually rotated to increase or decrease flow which can be monitored by viewing the drop rate of fluid moving through the drip chamber. In this view, the piston 34 is shown in the fully closed position. As cap 39 is rotated, threads 41 selectively advance or retract the position of the piston 34 within the cavity of flow resistor body 31, depending on the direction of rotation, exposing a helical channel or thread 37 to the incoming fluid, which enters the flow resistor body at inlet 33.

The groove 37 is made with an increasing pitch, width, and/or depth along its length, to selectively increase or decrease the flow area aligned with the inlet of the resistor, the taper of the pitch, width, and/or depth preferably being selected to create a logarithmically increasing flow path for the fluid as the resistor moves from the closed to fully open position. As the thread 37 is exposed to the fluid, fluid travels in the gap created by the threads 37 and cap 39 to flow into the space between cap 39 and piston 34. Fluid in this space exits the flow resistor through a central passage 38 in piston 34 to the outlet 32.

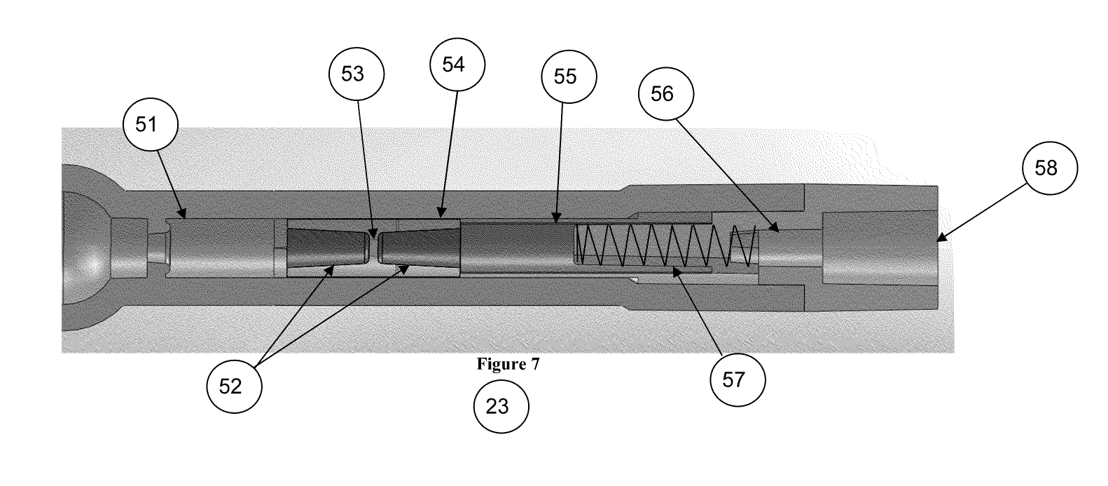

Piston 34 is sealed by an annular ring or protrusion 35 that slides in the cavity of the resistor body 31. Cap 39 is sealed by an O-ring 40. Note that when the cap 39 is rotated, there is no translation of cap 39 with respect to body 31. Rotation of cap 39 translates the piston 34, exposing or hiding different portions of the thread 37 to selectively increase or decrease fluid flow through the device. In contrast to mechanisms used in other systems, such as slide clamps and roller clamps, which when activated send a bolus of drug to the patient, movement of piston 34 does not in itself drive fluid. Therefore, no bolus of fluid to the patient can be created by opening the flow resistor. This unique feature adds yet another layer of safety to the patient and differentiates the device in this preferred embodiment. An exemplary fluid flow resistor may be as described in commonly-owned PCT application No. PCT/US2009/068349 filed Dec. 17, 2009, the entire contents of which are incorporated herein by reference. Fluid exiting variable flow resistor 22 via the outlet 32 enters flow sensor body 23 (see FIG. 7). A protrusion 36 rides in a corresponding groove 42 as the piston 34 is translated to prevent rotation of the piston 34 relative to flow axis.

Referring now to FIG. 7, fluid entering flow sensor body 51 is impeded by sensor element 52, held against the flow opening by spring 57. Sensor element 52 is generally opaque and houses a transparent transmitting element 53, which is transparent (as used herein, the terms transparent and opaque are used in reference to the wavelength of light emitted by the light array 13) and is designed to transmit light onto the sensor array 14. The transmitting element is preferably cylindrical and will be described herein primarily by way of reference thereto, however, it will be recognized that the focusing element 53 may be spherical, cylindrical, or other geometric configuration. An alternative embodiment, which has been contemplated, has a transmission region which is fundamentally spherical and thus focuses the transmitted light onto the sensor. In the alternative embodiment the transmitting element 53 may act a refractive lens, or may be a diffractive and/or holographic optical element for focusing light emitted by the array 13 onto the sensor array 14.

When disposable 16 is in controller 1, flow sensor 23 nests between light source array 13 and optical detector array 14 (see FIG. 2). Light emitted from array 13 is gathered by cylindrical element 53 and focused on detector array 14. As flow increases, sensor element 52 is displaced, compressing spring 57 seated at one end on spring seat 56. The interior flow channel 55 is tapered toward outlet 58 to allow higher flow as more of the tapered area is exposed by the displaced sensor element 52. Ribs 54 maintain sensor element 52 alignment with the central flow axis of the flow path.

There are various alternate embodiments that would be obvious to one skilled in the art, such as the use of a generally cylindrical transparent element in lieu of cylindrical element 53, allowing the transmission of light through the sensor to the detector without focusing the light. As would be understood by one skilled in the art, a sensor of this type when coupled with the light source array 13 and the optical detector 14 would produce unique output signals when measuring the passage of fluid as versus the passage of air. In addition, since air is compressible, bubbles generate a distinct output signal and the flow sensor herein can therefore additionally function as a bubble detector.

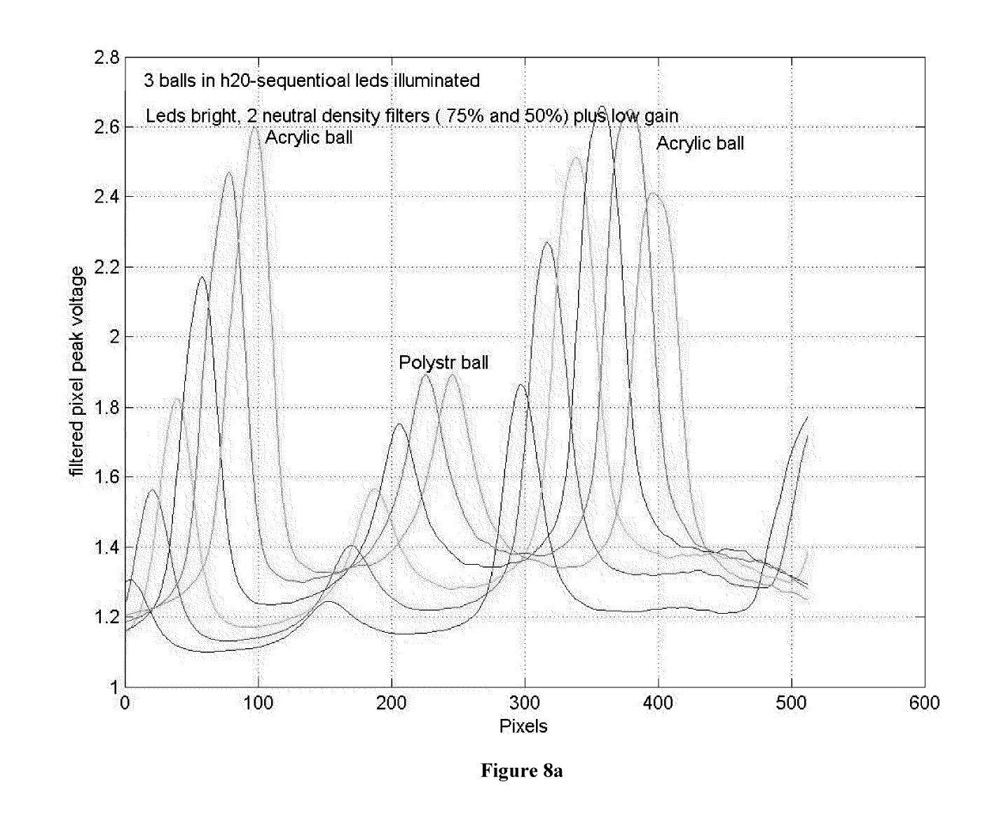

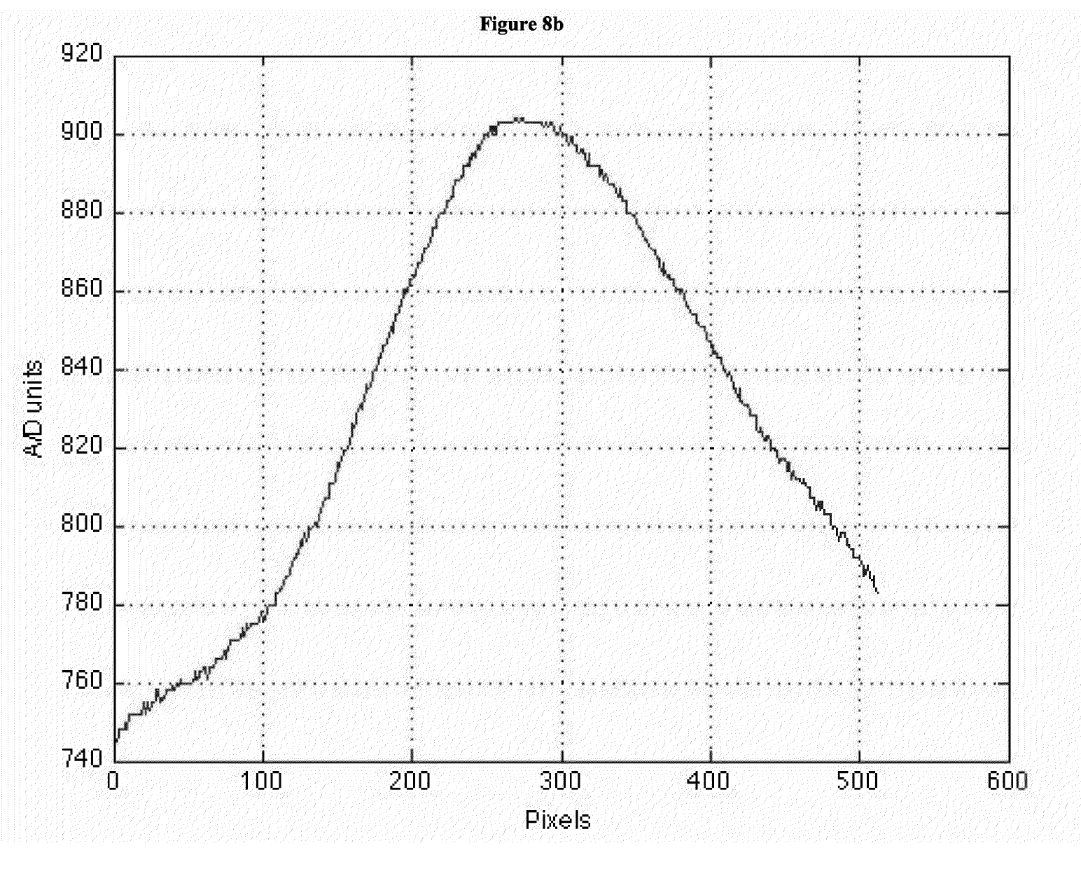

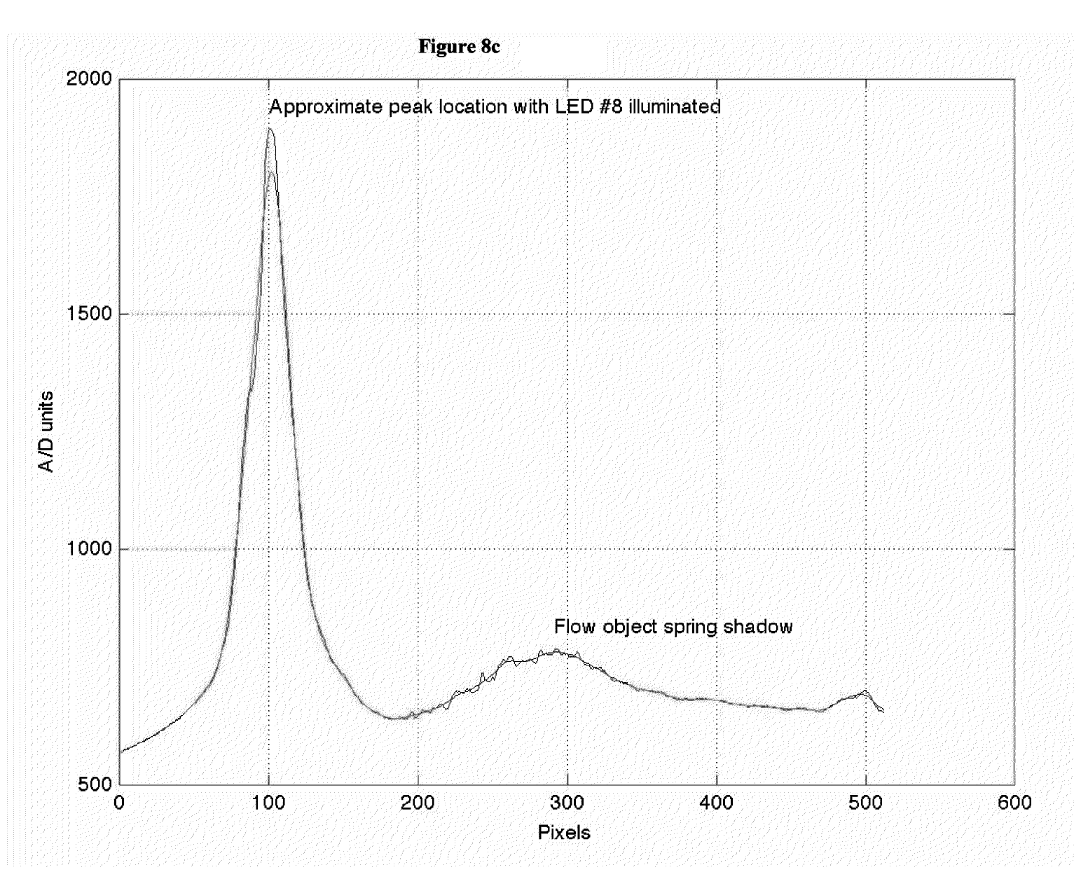

Referring now to FIGS. 8a-8c, it can be seen how significantly the signal voltage is enhanced by using a transparent cylindrical element to transmit light. Referring now to FIG. 8b, a graph is shown with a clear peak of the optical signal of the flow object. A graph showing a clear peak of the optical signal through TPN, a highly scattering fluid, is shown in FIG. 8c.

Referring again to FIG. 4, fluid passing through flow sensor 23 flows through tube 8 to the patient.

Referring now to FIG. 9, controller 1 mounts to pole mount 60 by means of the slide interface 4. Corresponding slides 61 receive controller 1. Low voltage DC electric power provided through cord 63 comes from a transformer connected to a standard AC outlet (not shown) and is transferred through the interface 4 and 61 to charge the batteries 112 of the device. Pole mount 60 can be clamped on any standard IV pole 62 and in the depicted embodiment supports up to four controllers.

A review of adverse infusion events on the FDA's reporting database (MAUDE) shows that a surprising number of adverse events occur each year as a result of a caregiver forgetting to plug the infusion pump back in after the pump or patient is moved. Other devices use only a tiny light or icon to show when the device is plugged in which can easily be missed. Subsequent battery alarms and battery failure can prevent the patient from timely receiving the medication prescribed.

The preferred embodiment of this system addresses this unmet need in two manners: first, pumping air to drive the infusion requires significantly less power than compressing a pumping segment with a peristaltic device, allowing for substantially longer battery life; and the device display will automatically go dark—an additional power savings feature—after a time out from input from a user or from sensed moving if it is not plugged in. The infusion will continue, and the display will periodically come to life, but this new behavior will alert the caregiver that the device is not plugged in and is significantly more prominent and therefore useful than a small indicator light or icon as commonly found on conventional devices.

Another source of adverse events present in other devices but not present in the preferred embodiment of this device is related to occlusions either upstream or downstream that prevent the infusion from proceeding as programmed. There are two associated hazards with other devices on the market with respect to occlusion detection: other devices depend on sensing pressure in the disposable to detect a no-flow condition. Pressure in the disposable will increase over time if there were a downstream occlusion as the pump would continue, filling the compliance available in the disposable until the pressure sensor is able to read sufficient pressure in the line to trip an alarm. When the occlusion is cleared (for example, when the line pinched when the patient was moved is straightened), the pressurized fluid in the line is delivered to the patient as a bolus. This can be a significant hazard as peristaltic pumps can generate high pressure (upwards of 15 psi) which, depending on the compliance of the set and associated delivery catheter and tubing can store and then immediately deliver a significant volume of drug.

The second hazard associated with pressure sensing as a secondary means of sensing fluid flow is that depending on the flow rate, the pressure alarm settings and the compliance of the tube set, the device can run for over two hours without delivering any medication before sufficient pressure builds in the set to trip the alarm. Some courses of therapy depend on a continuous infusion and a two hour interruption can be a significant source of concern. The preferred embodiment of the system disclosed senses flow directly, both with the flow sensor and with the pressure sensors in the intermediate pumping chambers (redundant flow sensing) and therefore is immediately aware of a no-flow condition regardless of the flow rate or the tubing compliance. Secondly, the pneumatic drive of the system typically operates at one psi, with a maximum of 5 psi available to drive an infusion—a huge improvement in safety as compared to pumps that can deliver fluid in excess of 15 psi.

Finally, the approach of the preferred embodiment allows for a significantly smaller, lighter, and more cost effective approach to accurately delivering an infusion because it does not require a precision mechanism. In instances where previously there had been a tradeoff in infusion delivery and cost, where infusion data, accuracy, and safety were traded off against the cost of delivering that infusion, the preferred embodiment shifts that economic model. In care situations that previously might use cost to drive the use of a gravity infusion or a simpler infusion device, the economics and simplicity of use of this approach allows the infusion to be given at a similar cost, with the advantages of improved safety and traceable electronic data records further reducing the cost of documentation.

While there has been shown and described what is considered to be preferred embodiments of the invention, it will of course, be understood that various modifications and changes in form or detail could readily be made without departing from the spirit of the invention. It is therefore intended that the invention be not limited to the exact forms described and illustrated, but should be construed to cover all modifications that may fall within the scope of the appended claims and their equivalents.

What is claimed is:

1. A system for the controlled delivery of medicinal fluid, the system comprising:
a flow control device including a processor, a source of pressurized gas, a first pressure sensor, position sensor, and a motor;
a fluid pathway assembly having an inlet, an outlet, a fluid pathway extending between said inlet and said outlet, and a variable flow resistor;
a pneumatic pumping chamber in said fluid pathway assembly, said pnuematic pumping chamber including a gas receiving volume, a fluid receiving volume, and a flexible membrane separating said gas receiving volume and said fluid receiving volume;
said source of pressurized gas fluidically coupled to said gas receiving volume for selectively increasing or decreasing a gas pressure within said gas receiving volume, wherein flow rate of the medicinal fluid during operation is responsive to pressure changes in said gas receiving volume;
said first pressure sensor fluidically coupled to said gas receiving volume for sensing a pressure in said gas receiving volume;
an inline flow sensor element received within said fluid pathway, said inline flow sensor element movable in response to a fluid flowing in said fluid pathway;
said position sensor for sensing a position of said inline flow sensor element in said fluid pathway, the position of said inline flow sensor element in said fluid pathway correlated to a rate of fluid flowing in said fluid pathway;
said motor coupled to said variable flow resistor to adjust said variable flow resistor wherein the rate of fluid flowing in said fluid pathway is responsive to adjustments to said variable flow resistor;
said processor for calculating a first calculated flow rate using pressure information from said first pressure sensor;
said processor for selectively adjusting one or both of the pressure within said gas receiving volume and said variable flow resistor during operation to achieve a target flow rate.

2. The system of claim 1, further comprising:
said processor for calculating a second calculated flow rate using a sensed position of said inline flow sensor element in said fluid pathway.

3. The system of claim 1, wherein said variable flow resistor is manually adjustable to regulate a rate of fluid flow in the fluid pathway assembly.

4. The system of claim 1, wherein the source of pressurized gas is a pump.

5. The system of claim 1, wherein the first calculated flow rate is calculated based on a change in a volume of gas contained within said gas receiving volume over time and further wherein the volume of gas contained within said gas receiving volume is calculated using an ideal gas law.

6. The system of claim 1, wherein said position sensor is calibrated using said first calculated flow rate.

7. The system of claim 1, further comprising:
said motor adjusting said variable flow resistor in accordance with the sensed signal from said position sensor representative of the position of said inline sensor element within said fluid pathway assembly.

8. The system of claim 1, further comprising:
said inlet fluidically coupled to a fluid source and said outlet fluidically coupling to a patient.

9. The system of claim 7, further comprising a visual indicator of fluid flow selected from one or both of a human viewable display and a drip chamber.

10. The system of claim 1, further comprising:
a display on said flow control device coupled to said processor for displaying flow rate information in human-viewable form.

11. The system of claim 1, further comprising:
said inline flow sensor element including an elongated element positioned to impede flow from the inlet to the outlet, said fluid pathway assembly further including a spring received within said fluid pathway to urge said elongated element in a direction opposite flow from said inlet, said elongated element further comprising a transparent spherical element centrally positioned in said elongated element; and
said position sensor including a light source and an optical detector for generating signals in response to movement of said spherical element.

12. The system claim 11, wherein said transparent spherical element acts as a lens to condition light transmitted from the light source to the optical sensor.

13. The system of claim 1, further comprising:
said inline flow sensor element including an elongated element positioned to impede flow from the inlet to the outlet, said fluid pathway assembly further including a spring received within said fluid pathway to urge said elongated element in a direction opposite flow from said inlet, said elongated element including an optically transparent element centrally positioned in said elongated element; and
said position sensor including a light source and an optical detector for generating signals in response to movement of said optically transparent element.

14. The system of claim 13, wherein said optically transparent element acts as a lens to condition light transmitted from the light source to the optical sensor.

15. The system of claim 1, further comprising:
at least one chamber of known volume
a second pressure sensor for sensing a pressure of said chamber of known volume; and
a valve for selectively isolating and fluidically coupling said chamber of known volume and said gas receiving volume.

16. The system of claim 15, wherein the volume of said pneumatic pumping chamber is not precisely known.

17. The system of claim 1, further comprising:
an inlet check valve for drawing the medicinal fluid from said inlet into said fluid receiving volume when a negative pressure is applied to said gas receiving volume and preventing passage of the medicinal fluid from said fluid receiving volume toward said inlet when a positive pressure is applied to said gas receiving volume; and
an outlet check valve for expelling the medicinal fluid from said fluid receiving volume to said outlet when a positive pressure is applied to said gas receiving volume and preventing passage of the medicinal fluid downstream of said fluid receiving volume into said fluid receiving volume when a negative pressure is applied to said gas receiving volume.

18. The system of claim 17, where said membrane seals an outlet of said fluid receiving volume preventing fluid flow when an increased gas pressure is maintained in said gas receiving volume.

19. The system of claim 15, further comprising:
said processor for calculating a volume of gas in said gas receiving volume using an initial pressure in the gas receiving volume and an initial pressure in the chamber of known volume when the gas receiving volume and the chamber of known volume are fluidically isolated, a final pressure in the gas receiving volume and the chamber of known volume when the gas receiving volume and chamber of known volume are fluidically combined, and a known volume of gas in the chamber of known volume.

20. The system of claim 19, further comprising:
said processor using periodic measurements of said volume of gas in said gas receiving volume to calculate said first calculated flow rate.

21. The system of claim 20, further comprising:
said processor comparing said first calculated fluid flow rate with a target flow rate and controlling said source of pressurized gas to change the pressure in said gas receiving volume until the first calculated fluid flow rate is substantially equal to the target flow rate.

22. The system of claim 20, further comprising:
said processor calibrating said position sensor by associating said first calculated flow rate with a sensed position of said inline flow sensor element in said fp.

23. A method for the controlled delivery of medicinal fluid from a fluid source to a patient, said method comprising:
connecting a first fluid source containing a first fluid to be infused into a patient to a first inlet of a fluid pathway assembly having a manually adjustable variable flow resistor, the fluid pathway assembly defining a fluid pathway and having a first pneumatic pumping chamber for driving fluid though the pathway and an inline flow sensor element received within the fluid pathway, the sensor element movable in response to fluid flowing in the fluid pathway;
connecting a fluid control device which is removably attachable to the fluid pathway assembly, the fluid control device providing the pneumatic control to drive fluid through the fluid pathway and having a sensor for detecting movement of the inline flow sensor element;
calculating an actual flow rate by one or both of measuring pressure changes in the pneumatic pumping chamber as a function of time or sensed position of the inline flow sensor element; and
monitoring the actual flow rate and automatically adjusting one or both of pressure in the pneumatic pumping chamber and the variable flow resistor to achieve a first target flow rate.

24. The method of claim 23, further comprising:
connecting a second fluid source containing a second fluid to be infused into a patient to a second inlet of said fluid pathway, said fluid pathway having a second pneumatic pumping chamber for driving fluid though the pathway.

25. The method of claim 23 further comprising:
calculating a difference between the actual flow rate and the first target flow rate;
if the difference between the actual flow rate and the first target flow rate is greater than a preselected threshold value, periodically varying a pressure in the pneumatic pumping chamber and measuring the actual flow rate until the difference between the actual flow rate and the first target flow rate is less than the preselected threshold value; and
if the difference between the actual flow rate and the first target flow rate is less than a preselected threshold value, periodically varying the pressure in the pneumatic pumping chamber and measuring the actual flow rate until the actual flow rate is equal to the first target flow rate.

26. The method of claim 25, further comprising:
providing the fluid pathway with at least one chamber of known volume, said chamber including check valve means to allow fluid in said fluid pathway to flow only from said inlet towards said outlet, said chamber divided with a flexible membrane moveable within said chamber and in fluidic contact with said first fluid to be infused to be delivered, said chamber including a port to pneumatically couple said control device to said chamber of said fluid pathway;
further providing means for varying gas pressure on said membrane through said port, said varying gas pressure causing said membrane to move within said chamber, expelling said first fluid to be infused from said chamber towards said outlet when the pressure is increased or drawing said first fluid to be infused from said inlet when the pressure is decreased; and
monitoring the actual flow rate and automatically adjusting the either or both of the pneumatic profile on the membrane and the variable flow resistor to achieve a first target flow rate.

27. The method of claim 25, further comprising:
monitoring a signal provided by the flow sensor and triggering an alarm if the signal indicates the passage of air.

28. The method of claim 23, further comprising:
monitoring the actual flow rate and triggering an alarm if the actual flow rate is unable to match the first target flow rate.

29. The method of claim 23, further comprising:
communicating infusion status information to a computer based information handling system via a wireless communication link.

30. The method of claim 29, further comprising:
automatically detecting device location and communicating location information to the computer based information handling system.

31. The method of claim 30, further comprising:
determining if the location of the device is associated with a patient identifier;
if the location of the device is associated with a patient identifier, associating the device with the patient identifier.

32. The method of claim 30, where the wireless communication link is accomplished through a mesh network.

33. The method of claim 29, further comprising:
automatically detecting caregiver identification and communicating caregiver identification to the computer based information handling system.

34. A system for the controlled delivery of medicinal fluid, the system comprising:
a first medicinal fluid to be delivered;
a fluid pathway assembly defining a fluid pathway and having an inline flow sensor element received within said fluid pathway, said inline flow sensor element movable in response to a fluid flowing in said fluid pathway;
a flow control device removably attached to said fluid pathway assembly and having a position sensor for detecting movement of said inline flow sensor element;
said fluid pathway assembly having a variable flow resistor which is manually adjustable to regulate a rate of fluid flow in the fluid pathway assembly;
said fluid pathway including a first chamber including a first chamber volume and a second chamber volume separated by a first flexible member intermediate the first chamber volume and the second chamber, the first chamber volume being filled with said first medicinal fluid to be delivered and the second volume filled with gas and including a port;
check valves for controlling flow into and out of said first chamber;
said flow control device including means for pneumatically coupling a chamber of known volume to said port in said second chamber volume, said flow control device further including means to selectively increase or decrease the pressure in said second chamber volume, said increase or decrease in pressure acting to move said first flexible member in said chamber to selectively expel the first medicinal fluid from the first chamber volume or draw the first medicinal fluid into said first chamber volume;
a motor attached to the flow control device, said motor operably coupled to said variable flow resistor when said flow control device is attached to said fluid pathway assembly; and
said variable flow resistor adjustable by said motor under automatic control to adjust a rate of flow of said first medicinal fluid in said fluid pathway.

35. The system of claim 34, further comprising:
means to measure a pressure of said gas in said second chamber volume;
means to determine a volume of the medicinal fluid present in said first chamber volume based on said pressure of said gas in said second volume; and
means for periodically calculating the volume of the medicinal fluid in said first chamber volume over time to determine a measured flow rate.

36. The system of claim 35, further comprising:
means for modifying the pressure in said second chamber volume until the measured flow rate matches a preselected target flow rate.

37. The system of claim 34, further comprising:
said position sensor for detecting movement of said inline flow sensor element including a light source and an optical detector;
said inline flow sensor element received within said fluid pathway including a flow element positioned in a tapered portion of the flow path;
said inline flow sensor element further including a spring to urge said flow element to oppose flow from said inlet; and
said light source and said optical detector positioned around said flow element to detect a position of the flow element when the force of fluid flow causes said flow element to move towards said outlet, compressing said spring.

38. The system of claim 37, wherein said flow element includes a spherical element to focus said light to improve resolution on said detector.

39. The system of claim 35, further comprising means to wirelessly communicate infusion status information to a computer based information handling system.

40. The system of claim 39, wherein the computer based information handling system is a network.

41. The system of claim 40, wherein said network is a mesh network.

42. The system of claim 41, wherein said flow control device include means to automatically detect a location of the system.

43. The system of claim 34, further comprising:
means for determining when said position sensor for detecting movement of said inline flow sensor element indicates the passage of air; and
means for generating an alarm when said air passage is detected.

44. The system of claim 35, wherein said measured flow rate is used to calibrate the output of said position sensor for detecting movement of said inline flow sensor element.

45. The system of claim 34, further comprising:
a second medicinal fluid to be delivered, said fluid pathway including a second inlet;
said second medicinal fluid being fluidically connected to said second inlet;
said second inlet including a pathway to a second chamber including a third chamber volume and a fourth chamber volume separated by a second flexible membrane, said third chamber volume being filled with said second medicinal fluid to be delivered and said fourth chamber volume filled with gas and including a second port;
check valves for controlling flow into and out of said second chamber;
said flow control device including means for pneumatically coupling the chamber of known volume to said second port in said second chamber, said device further including a means to selectively increase or decrease the pressure in said fourth chamber volume, said increase or decrease in pressure acting to move said flexible member in said second chamber to selectively expel the second medicinal fluid out of or draw the second medicinal fluid into said third chamber volume; and
means to preferentially modify pressure in either said second chamber volume or said fourth chamber volume to selectively deliver said first or said second medicinal fluid to be delivered.

46. The system of claim 44, further comprising a program of instructions for:
sensing an abrupt flow increase from the flow sensor;
measuring a duration of the abrupt flow increase;
integrating the sensed abrupt flow increase; and
creating an electronic record of a possible upstream manual bolus.

47. The system of claim 46, further comprising:
a user interface for generating output prompting a caregiver to confirm and identify the possible upstream manual bolus.

48. The system of claim 47, further comprising:
said user interface for receiving input from the caregiver for updating the electronic record based on said input.
